(19) United States
(12) Patent Application Publication
(10) Pub. No.: US 2007/0093859 A1
(43) Pub. Date: Apr. 26, 2007

(54) Suture passer device for abdominal or thoracoscopic surgery

(76) Inventor: Edward H. Phillips, Beverly Hills, CA (US)

Correspondence Address: Frederick Gotha, Suite 823, 80 South Lake Avenue, Pasadena, CA, 91101, US

(21) Appl. No.: 11/259,825

(22) Filed: Oct. 26, 2005

(51) Int. Cl. A61B 17/04

(52) U.S. Cl. 606/148

(57) ABSTRACT

This invention relates to an improved suture passer device for inserting and retrieving suture during abdominal or thoracic surgery. The device consists of a probe member having a blunt needle tip that forms the terminal distal end of the probe member. A flexible thread having a bight portion is so carried by the probe member to permit the bight portion to extend from a lateral port located intermediate the distal terminal end and proximate end of the probe member. The extension of the bight portion through the lateral port forms a loop for receiving an end segment of the suture.

### FIELD OF THE INVENTION

This invention relates to a medical instrument for passing and retrieving suture through the abdominal and thoracic regions during abdominal or surgical procedures.

### BACKGROUND OF THE INVENTION

Endoscopic surgery of the abdominal and thoracic regions involves the placement of ports through tocar incisions made in the abdominal and thoracic regions. Typically, for procedures such as a laparoscopic procedure, the surgery is performed under carbon dioxide pneumoperitoneum which is obtained through a Veress needle introduced by a micro incision in the abdominal wall. A video camera is then positioned through a port for general intra-abdominal assessment where the intra-peritoneal organs may be thoroughly examined. Likewise, for a thoracic procedure, a thoracoscope or video camera may be introduced through a small thoracic incision into the thoracic region for examination of the pleural cavity; other instruments may be introduced through other small thoracic incision ports into the pleural cavity. After the surgical procedure is completed, the incisions which permitted the introduction of the trocars and surgical instruments into the body cavity must be sutured closed to unite the opposite surfaces of the incision. Suture passers were utilized in the prior art to introduce and pass the suture through the abdominal wall into the cavity and then captured endoscopically in the cavity. The suture passer then was passed through a different pre-selected site where the site was selected so as to permit a stitch to be made to unite the opposite surfaces of the incision; it was also used when performing laparoscopic mesh repair of abdominal wall hernias which required the passage of suture through the abdominal wall. Suture passers of the prior art utilized an open needle-blunt tipped probe member where a loop of flexible thread extended axially through a radial opening in the terminal tip of the probe. The terminal tip radial opening offered erratic resistance to the passage of the probe through the fascia which packed into the tip opening thereby decreasing the effectiveness of the needle and increasing the possibility of tearing the tissue and blood vessels leading to bleeding and pain. The present invention utilizes a closed blunt needle tip where the opening through which the flexible loop extends is located at a distance axially removed from the terminal tip.

### SUMMARY OF THE INVENTION

There is, therefore, provided according to the present invention an improved suture passer device that has a blunt needle tip forming the terminal end of a probe member and having a lateral port axially and proximately removed from the distal terminal end that permits a bight or flexible loop to extend laterally therefrom. The improved suture passer therefor enters through the fascia with less resistance and reduced probability of the tissue tear as the flexible loop is carried therethrough.

The present invention in its preferred embodiment is directed to a improved suture passer device for inserting and retrieving suture during abdominal or thoracic surgery. In the preferred embodiment, the improved suture passer consists of a probe member having an axis of elongation, a distal blunt needle tip forming the terminal end of the probe member, and a lateral port located proximately from the terminal end. A flexible thread having a bight portion is so adapted and carried by the probe member to permit the bight portion to extend from the lateral port forming a loop for receiving an end segment of the suture; the end segment of the suture is passed through the fascia by insertion of the tip of the probe member into a body cavity.

In another embodiment, the probe member is of a known or typical construction of the prior art; namely, it has an axis of elongation, a distal tip forming the terminal end of the probe member, and a radial opening in the terminal end of the distal tip. A flexible thread having a bight portion is so adapted and carried by the probe member to permit the bight to extend axially through the radial opening forming a loop. The improvement consists of a sleeve having an axially extending cavity and an open proximate end communicating with the cavity. The sleeve has a blunt needle tip forming its distal terminal end where the sleeve is removably and telescopically carried by the probe member. The sleeve has an axially extending slot intermediate the proximate and distal ends of the probe member where the slot is so dimensioned and proportioned to permit the bight of the flexible thread to extend through the slot forming a loop external to the sleeve. The end segment of the suture is received by the loop which is then passed through the fascia by insertion of the sleeve needle tip into the body cavity.

### BRIEF DESCRIPTION

These and other features and advantages will become appreciated as the same become better understood with reference to the following specification, the claims and drawings wherein:

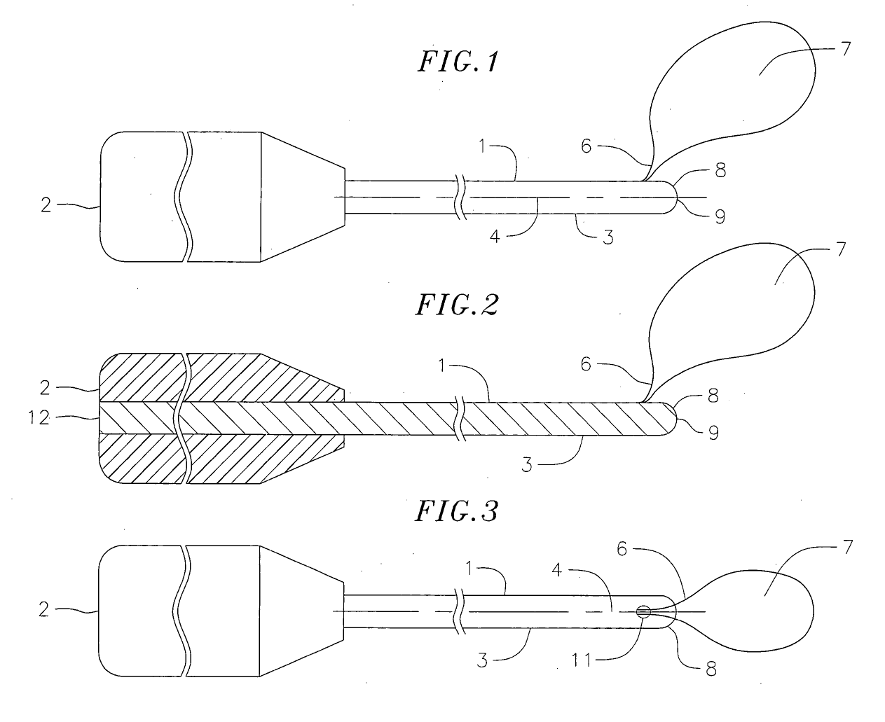

FIG. 1 is a side view of the preferred embodiment of this invention;

FIG. 2 is a side cross-sectional view of FIG. 1;

FIG. 3 is a top view of FIG. 1;

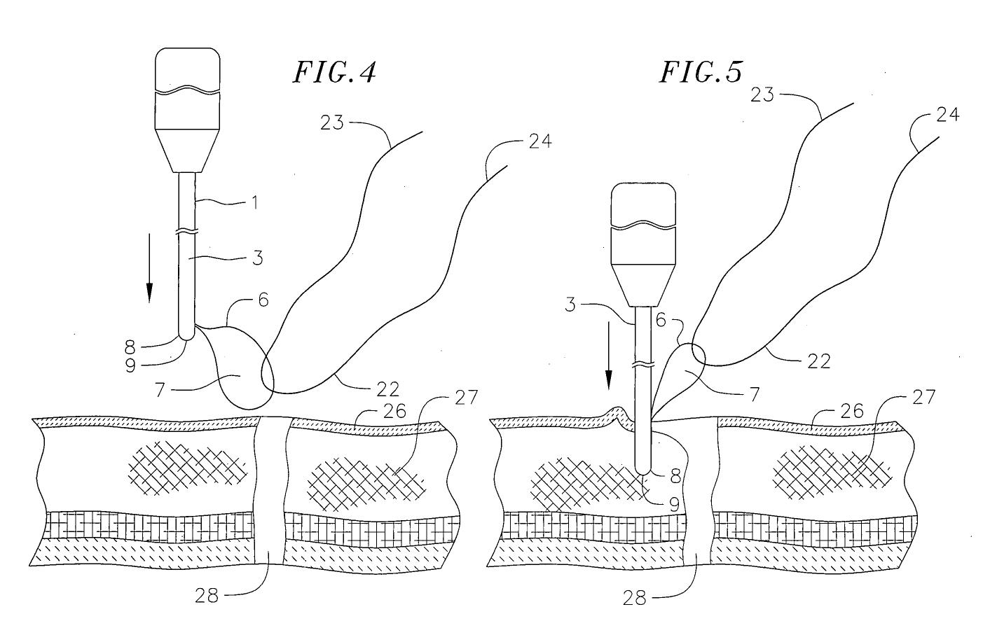

FIG. 4 is an illustrative view showing an incision extending through the outer skin layer and fascia to a body cavity with a segment of suture inserted through the bight portion of a flexible thread;

FIG. 5 is an illustrative view of the improved suture passing device of this invention illustrating the passage of the terminal tip of the suture passer into the fascia;

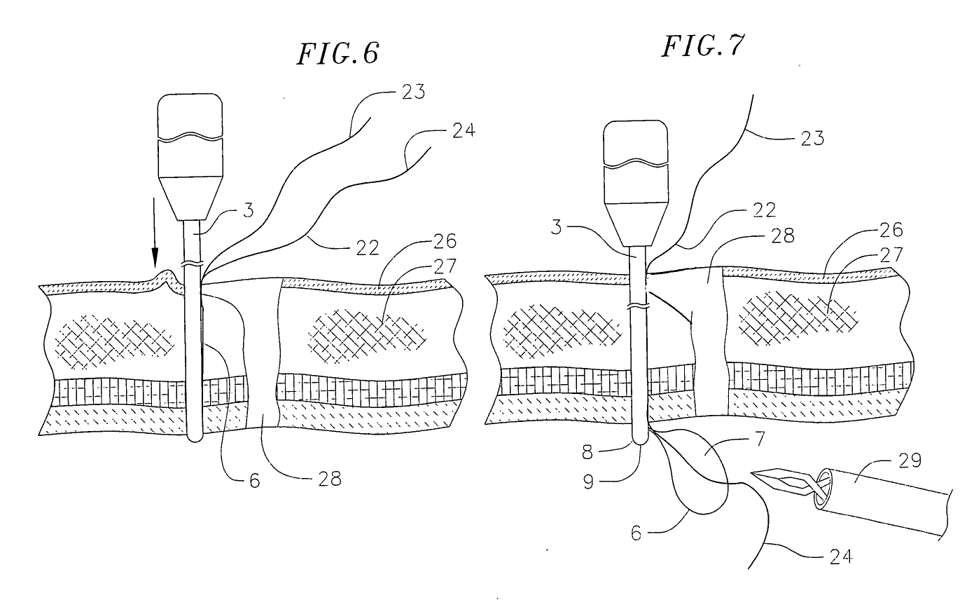

FIG. 6 illustrates the passage of the terminal tip of the improved suture passer through the fascia and emerging into a body cavity;

FIG. 7 illustrates the passage of the bight or loop of the flexible thread and the segment of suture into the body cavity where the suture segment may be grasped by a grasper instrument within the cavity;

FIG. 8 illustrates the end segment of the suture grasped by the grasper instrument;

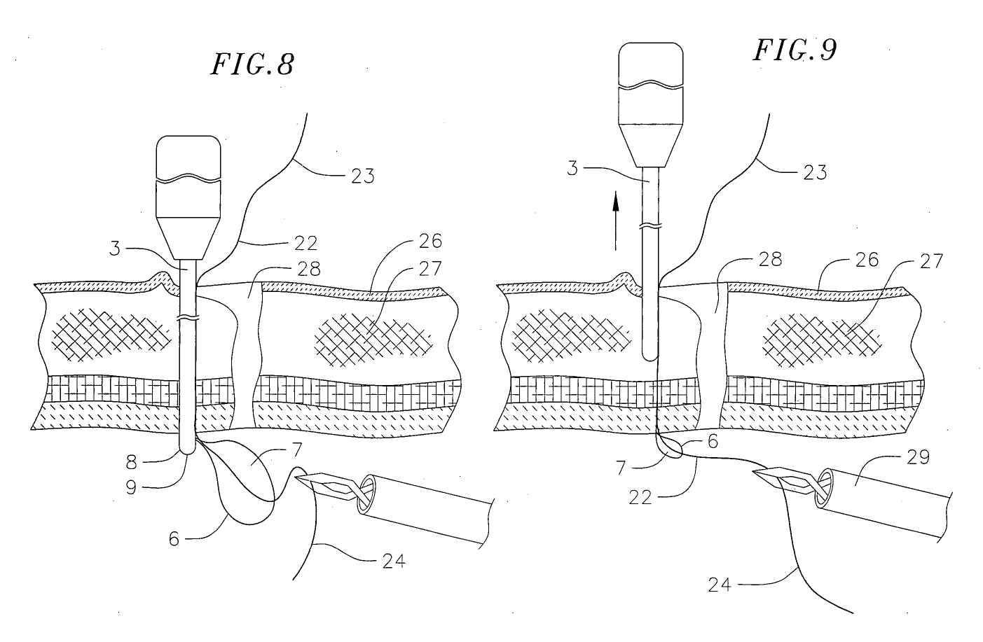

FIG. 9 illustrates the withdrawal of the improved suture passer through the fascia;

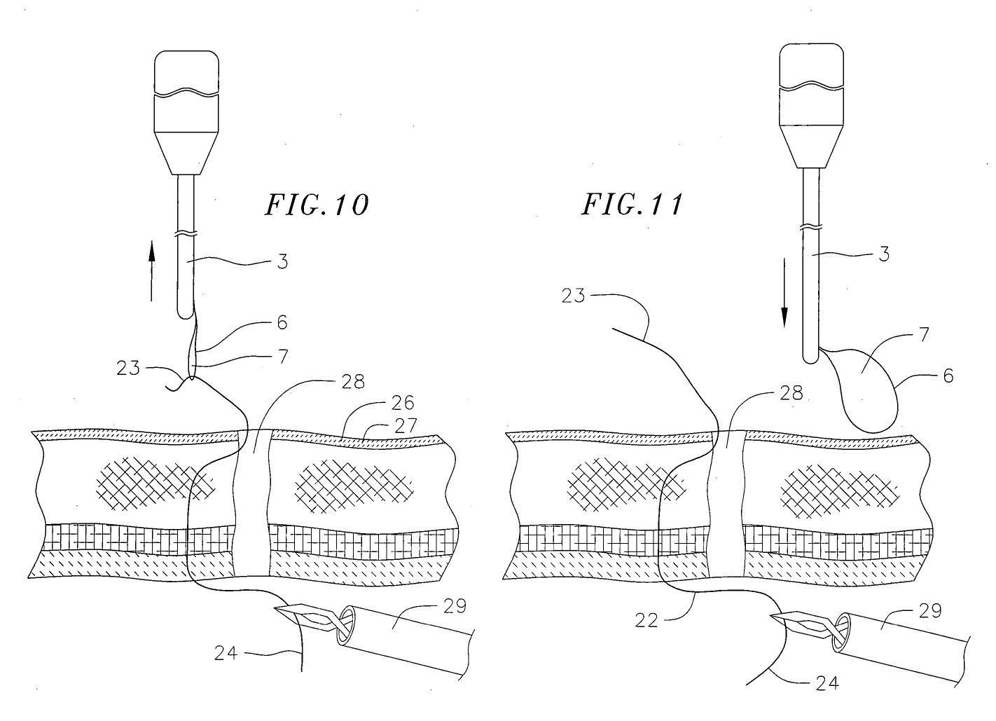

FIG. 10 illustrates the complete withdrawal of the improved suture passer with the flexible thread loop external to the body carrying an end segment of the suture;

FIG. 11 illustrates the improved suture passer positioned on the opposite side of the incision for passage through the fascia as illustrated in FIGS. 5, 6, 7, 8, 9, and 10;

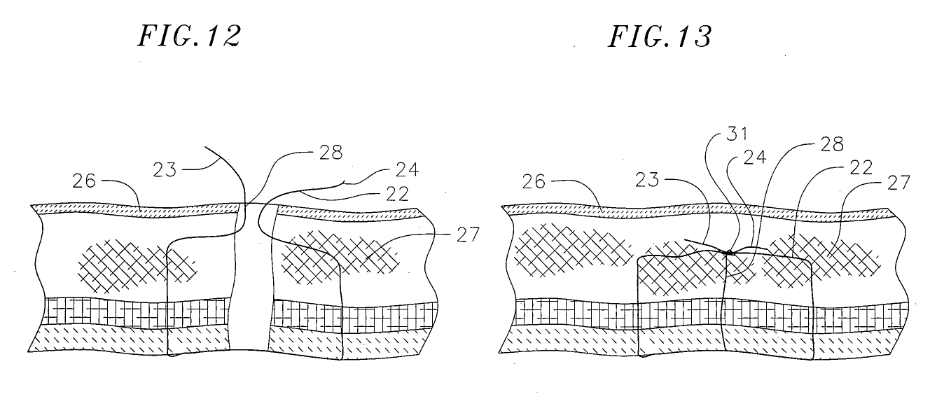

FIG. 12 illustrates the end segments of the suture on opposite sides of the incision before the incision is closed;

FIG. 13 illustrates the closing of the incision by the suture uniting the opposite surfaces of the incision;

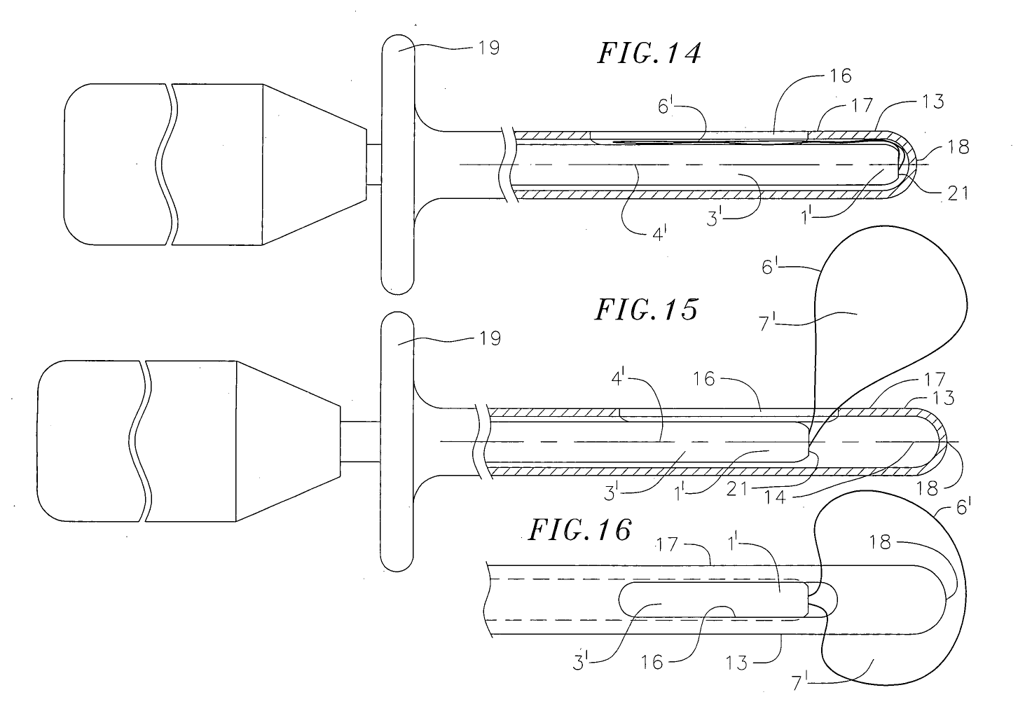

FIG. 14 is a side cross sectional view of an alternative embodiment of this invention illustrating a sleeve in telescopic relationship with a conventional suture passer;

FIG. 15 is a side cross sectional view of the alternative embodiment of FIG. 14 illustrating the loop of the conventional suture passer extending through the sleeve; and

FIG. 16 is a top view of FIG. 15.

### DETAILED DESCRIPTION

FIGS. 1 and 2 illustrate a side view of the preferred embodiment of the improved suture passer device of this invention. As can be seen in FIGS. 1 and 2, the suture passer device 1 has a handle 2, a probe member 3, an axis of elongation 4, and a flexible thread 6 forming a bight or loop 7. Probe member 3 is preferably made of metal and has a blunt needle tip 8 forming the terminal distal end 9 of probe member 3. The bight or loop 7 of flexible thread 6 extends through lateral port 11 (shown in FIG. 3) and opposing end segments of flexible thread 6 (not shown) are carried in fixed relationship with probe member 3. Lateral port 11 is so dimensioned and proportioned to permit the loop portion 7 of flexible thread 6 to extend externally of probe member 3 and articulate omnidirectionally in relationship to axis of elongation 4. In the preferred embodiment, lateral port 11 is axially removed from the distal terminal end 9 and located intermediate the proximate end 12 of probe member 3 with the end segments of flexible thread 6 fixed to the probe member 3. However, the end segments may be secured to the probe member 3 lateral surface such that the thread is carried on the lateral surface of the probe.

Another embodiment of this invention is illustrated in FIGS. 14, 15, and 16. As can be seen in these figures, a sleeve member 13 telescopically engages probe member 3¹. Sleeve member 13 has an axially extending cavity 14 that communicates with slot 16 which extends at least in part axially along the lateral surface 17 of sleeve member 13. The distal terminal tip 18 of sleeve member 13 is a blunt needle for inserting below the outer skin layer 26 and through the fascia 27 into the body cavity. Sleeve member 13 is preferably made of metal and has a base 19 which is cylindrically shaped with the axis of the cylinder concentric with the elongation axis 4¹ of probe member 3¹.

The suture passer 11 shown in FIGS. 14, 15, and 16 is a conventional device that is presently used and known in the prior art. As can be seen in these figures, suture passer 11 has a radial opening 21 forming the terminal distal tip of probe member 3¹. Flexible thread 6¹ extends axially through radial opening 21 forming the bight or loop 7¹. Thus, when sleeve member 13 telescopically and slidably engages probe member 3¹ as shown in FIG. 14, flexible thread 6¹ is collapsed in an axial proximate direction from terminal tip 18. When sleeve member 13 is positioned as shown in FIGS. 15 and 16, the bight or loop 7¹ will extend through slot 16 externally of the lateral outer surface 17 of the sleeve. The configuration of this embodiment after the sleeve is positioned as shown in FIGS. 15 and 16, permits the embodiment to function in the same manner as the preferred embodiment. The operation of both the preferred and other embodiment is shown in FIGS. 4-13 and is hereafter described in detail.

Referring to FIGS. 4 and 5, suture 22 has a first end segment 23 and a second end segment 24 where an end segment of the suture 22 is extended through loop 7 to permit trailing of the suture 22 as the suture 22 by passes the epidermis or outer skin layer 26 and enters into the fascia 27 on its passage into the body cavity on one side of incision 28; the arrow in FIGS. 4 and 5 indicates the direction of suture passer 1 in its passage into the body. As can be seen in FIG. 5, the needle tip 8 forming terminal end 9 of probe member 3 has entered into the fascia 27; the continued passage of probe member 3 through the fascia 27 is illustrated in FIGS. 6 and 7. The emergence of the distal terminal end 9 into the body cavity of probe member 3 and complete passage of loop 7 into the body cavity can be seen in FIG. 7. Within the body cavity, a grasper instrument 29 is positioned by the surgeon to grasp end segment 24 of suture 22. The sequence of closing incision 28 is further illustrated in FIGS. 8, 9, 10, 11, 12, and 13.

In FIG. 8, the end segment 24 is grasped by grasper instrument 29 and held; the probe member 3 is then withdrawn as shown in FIG. 9 and, as can be seen in FIG. 10, end segment 23 is held externally to the body by loop 7 after probe member 3 is withdrawn through the fascia 27 and past outer skin layer 26. The probe member 3 is then re-inserted to grasp end segment 24 of suture 22 and retrieve it through fascia 27 on the opposite side of incision 28; the beginning of this sequence is shown in FIG. 11. After loop 7 is passed into the body cavity, grasper instrument 29 is manipulated by the surgeon laparoscopically to grasp second end segment 24 and pass the end segment through loop 7. The probe member 3 is then withdrawn through the fascia 27 and past the outer skin layer 26; the end segments 23 and 24 are now positioned on opposite sides of incision 28 (as can be seen in FIG. 12). To unite the opposite sides of incision 28, as shown in FIG. 13, a stitch 31 is made by the surgeon to close incision 28.

The procedure described above for the preferred embodiment of this invention to introduce the suture 22 to opposing sides of incision 28 is identical for the alternative embodiment described in FIGS. 14, 15, and 16.

While I have shown and describe embodiments of an improved suture passer, it is to be understood that the invention is subject to many modifications without departing from the scope and spirit of the claims as recited herein.

What is claimed:

1. A suture passer device for inserting and retrieving suture during abdominal or thoracic surgery, comprising:
(a) a probe member having an axis of elongation, a distal tip forming a distal terminal end of said probe member, and a lateral port located adjacent said distal terminal end; and
(b) a flexible thread having a bight portion where said flexible thread is so adapted and carried by said probe member to permit said bight portion to extend through said lateral port forming a loop for receiving said suture.

2. The suture passer device recited in claim 1 where said probe member has a proximate end and said lateral port is located intermediate said proximate end and said distal terminal end.

3. The suture passer device recited in claim 1 where said distal tip is curved.

4. The suture passer device recited in claim 1 where said flexible thread has a finite length with a first end portion and a second end portion, said first and second end portions carried in fixed relationship with said probe member.

5. The suture passer device recited in claim 1 where said probe member is made of a metal material.

6. An improved suture passer device having a probe member, an axis of elongation, a distal tip, a radial opening in said distal tip forming the distal terminal end of said probe member, a flexible thread carried in fixed relationship with said probe member having a bight portion extending axially through said radial opening forming an external loop for receiving the end segment of a suture, the improvement comprising a sleeve member having an axis of elongation, a proximate end and a distal terminal tip, an axially extending cavity bounded by said distal terminal tip, where said proximate end of said probe member has a radial opening communicating with said cavity, and where said cavity is so dimensioned and proportioned to receive said probe member in telescopic and slidable relationship with said sleeve, said sleeve further having an axially extending slot intermediate said proximate end and said distal terminal tip communicating with said cavity such that upon sufficient axial displacement of said sleeve, said bight portion is extendable through said slot.

7. The improved suture passer device recited in claim 6 where said distal terminal tip of said sleeve member is curved.

8. The improved suture passer device recited in claim 6 where said axis of elongation of said probe member and said axis of elongation of said sleeve member are co-axial.

9. The improved suture passer device recited in claim 6 where said sleeve member is made of a metal material.

10. A suture passer device comprising in combination:
(a) A probe member having a proximate end and a distal tip, an axis of elongation, a radial opening in said distal tip forming the distal terminal end of said probe member, a flexible thread carried in fixed relationship with said probe member, said flexible thread having a bight portion extending axially from said radial opening forming a loop for receiving the end segment of said suture; and
(b) a sleeve member having an axis of elongation, a proximate end and a distal terminal tip, an axially extending cavity bounded by said distal terminal tip, where said proximate end of said probe member has a radial opening communicating with said cavity, and where said cavity is so dimensioned and proportioned to receive said probe member in telescopic and slidable relationship with said sleeve, said sleeve further having an axially extending slot intermediate said proximate end and said distal terminal tip communicating with said cavity such that upon sufficient axial displacement of said sleeve, said bight portion is extendable through said slot.

11. The combination recited in claim 10 where said distal terminal end of said sleeve is curved.

12. The combination recited in claim 10 where said axis of elongation of said probe member and said axis of elongation of said sleeve member are co-axial.

13. The combination recited in claim 10 where said sleeve member is made of a metal material.
